(19) United States
(12) Patent Application Publication
(10) Pub. No.: US 2024/0409863 A1
(43) Pub. Date: Dec. 12, 2024

(54) PROCESS FOR PRODUCING ALCOHOLS BY FERMENTATION

(71) Applicant: IFP ENERGIES NOUVELLES, Rueil-Malmaison (FR)

(72) Inventors: Maxime CARRIE, Rueil-Malmaison Cedex (FR); Helene VELLY, Rueil-Malmaison Cedex (FR); Jean-Christophe GABELLE, Rueil-Malmaison Cedex (FR); Mohamed Fadhel BEN CHAABANE, Rueil-Malmaison Cedex (FR)

(73) Assignee: IFP ENERGIES NOUVELLES, Rueil-Malmaison (FR)

(21) Appl. No.: 18/703,050

(22) PCT Filed: Oct. 10, 2022
(86) PCT No.: PCT/EP2022/078032

§ 371 Date: Apr. 19, 2024

(30) Foreign Application Priority Data
Oct. 20, 2021 (FR) FR2111141

(51) Int. Cl. C12M 1/00; C12M 1/12; C12M 1/34; C12M 1/36; C12P 7/06; C12P 7/16; C12R 1/145

(52) U.S. Cl. CPC C12M 21/12; C12M 25/14; C12M 41/34; C12M 41/36; C12M 41/48; C12P 7/065; C12P 7/16; C12R 2001/145

(57) ABSTRACT

The present invention relates to a process for producing alcohol(s), according to which a culture medium containing a sugary carbon substrate (2) is introduced into a reaction section (1) comprising a support (4) on which micro-organisms are immobilised, in order to produce, by fermentation under the action of the said micro-organisms, a must (3) enriched with alcohol(s) and fermentation gas(es), such that the process is operated continuously in the liquid phase, and such that, in order to control production, the quantity of living micro-organisms immobilised on the support is monitored without intervention on the said supports.

### TECHNICAL FIELD

The present invention relates to a process for producing alcohols by continuous fermentation, with immobilized cells, of a culture medium containing a sugary carbon-based substrate.

### PRIOR ART

In order to meet the energy transition challenges, considerable research is being conducted to develop “green” processes, affording access to chemical intermediates in an alternative manner to the refining of petroleum and/or petrochemistry.

Alcohols derived from fermentation processes (for example isopropanol and n-butanol) are among the most promising replacements for petrochemical derivatives. ABE (Acetone-Butanol-Ethanol) fermentation, performed by microorganisms belonging to the genus Clostridium, is one of the oldest fermentations to have been industrialized, and has since been extensively studied. More recently, IBE (Isopropanol-Butanol-Ethanol) fermentation, producing a mixture of isopropanol, butanol and ethanol and also performed by solventogenic microorganisms belonging in particular to the genus Clostridium, has been the subject of numerous studies.

As regards the fermentation approach employed in this type of process, batch production remains the conventional method for ABE and IBE fermentations, despite the low productivity displayed for this type of process, in the range 0.1-0.7 g/L·h (see, for example, Jones D. T., Woods D. R., 1986, Acetone-Butanol Fermentation Revisited. Microbiol. Rew., 50 (4), 484-524 or Table 16.6 Lopez-Contreras A. et al. chapter book 16, Bioalcohol Production: Biochemical Conversion of Lignocellulosic Biomass, 2010). However, these productivities remain too low to envisage an economically viable industrial process.

A continuous process with cells in suspension in a homogeneous reactor may also be envisaged. However, the productivity is also relatively low and cannot easily be significantly increased. One technical problem is the concentration of the cells in the fermentation medium, which is mainly controlled by the dilution rate applied in the process. This rate cannot be high, to avoid cell “wash-out” in the bioreactor.

For these reasons, great interest has been shown in recent years in methods directed toward high retention of the microbial biomass, in particular by immobilization of the microorganisms in the bioreactor. The use of a continuous process with immobilized cells enables a significant increase in the volume productivity of alcohol, since the residence time of the microorganisms under these conditions is decorrelated from the hydraulic residence time of the bioreactor under study. Moreover, the concentration of microorganisms is higher in the bioreactor.

The present invention will thus more particularly focus on the technique for immobilizing the cells: A process has thus been proposed in patent FR-3 086 670 in which at least a portion of the bacterial biomass is fixed in the fermentation reactor by adsorption in the form of a biofilm on a porous material based on polymeric foam, of polyurethane foam type. This material has proven to be particularly efficient, allowing a continuous fermentation process, the foam making it possible to fix the bacteria in a sufficiently substantial manner, i.e. beyond the dilution rate causing cell wash-out. This material opens up a new pathway for the production of mixtures of IBEA type, while also giving access to a production in continuous mode by immobilization of the bacterial biomass.

It is useful, for the performance of the fermentation process, to evaluate the efficacy of the microorganisms immobilized on supports in the fermentation reactor. This is because the microorganisms are immobilized in the form of a biofilm on the solid support. This biofilm is composed of a mixture of microorganism cells and extracellular polymers. The process of biofilm formation is not readily controllable and depends on numerous factors. Specifically, the hydrodynamic conditions in the bioreactor, the physicochemical properties of the supports used and also the number thereof, in particular, can influence biofilm development. The proportion of viable, and hence metabolite-producing, cells within this structure can also vary widely depending on the age of the biofilm under study and also on the operating conditions of the process. In addition, the immobilized microorganisms tend to produce butanol, which is a compound that inhibits the growth of the microorganisms, which results in having to increase the incoming volume flow rate of the fluid containing the sugars to be fermented (and outgoing flow rate of the fluid containing the fermentation products).

For all of these reasons, it has been found that monitoring the total quantity of living microorganisms in the biofilm which is deposited on the supports in the bioreactor is a key parameter for optimally managing the fermentation process. However, it is difficult to monitor this. Specifically, it is difficult to envisage sampling of the supports outside of the bioreactor, since the bioreactor generally operates with extreme conditions of sterility and anoxia which would be significantly disrupted by the regular opening of the bioreactor for taking these samples. Moreover, it is complicated to have a representative sample from which the total concentration of viable cells could be quantified. Lastly, the extraction methods take time and may introduce a significant bias to the measurement.

In addition, studies have focused on means for estimating the rate of growth of the biofilms, notably via the electrochemical route, as described in particular in the patent application EP-3 035 052, without however succeeding in estimating the proportion of living microorganisms in these biofilms. In addition, the implementation of these solutions is complex in an industrial-scale installation.

The aim of the invention is thus to overcome these drawbacks. An aim of the invention is in particular to develop fermentation processes utilizing microorganisms immobilized on supports, which are easier to control. An aim of the invention in particular is to more easily and/or more precisely quantify the change in activity of these immobilized microorganisms.

### SUMMARY OF THE INVENTION

A subject of the invention firstly is a process for producing alcohol(s), according to which a culture medium containing a sugary carbon-based substrate is introduced into a reaction section comprising a support on which microorganisms are immobilized, in order to produce, by fermentation under the action of said microorganisms, a must enriched in alcohol(s) and one or more fermentation gases (CO₂/H₂), such that the process is operated continuously in liquid phase, and such that, in order to control the production, the quantity of living microorganisms immobilized on the support is monitored without intervention on said supports.

The term “reaction section” is understood to be at least one bioreactor together with all the equipment making it possible to carry out a fermentation. If the reaction section comprises a plurality of bioreactors, these may be operated in parallel and/or in series. Some of them may be operating while one or more others are undergoing maintenance, in particular for changing the immobilization supports, in order not to interrupt production.

The term “support” is understood to mean a material, for example of the type as described in the abovementioned patent FR-3 086 670, on the walls of which the microorganisms can deposit (and progressively form biofilms), with reference then being made to immobilized microorganisms. It is generally a porous material, which may be of polymeric nature, such as a foam of polyurethane-based foam type, or of mineral nature, such as a porous material of ceramic type, etc. It may be in monobloc form, or in the form of several blocks arranged in an ordered manner (in superposed layers, for example) or unordered manner (loose) in the volume of the reaction section in which the fermentation is carried out. These blocks may be of regular geometric form and have the same size (cubes for example), or be of irregular form and/or have different sizes.

The expression “without intervention on said supports” is understood to refer to the fact that the invention does not involve any manipulation of the supports, in particular that it avoids taking samples of support outside of the reaction section, in particular in view of analyzing them. This means that the invention is implemented without opening the reaction section, without opening the bioreactor(s) comprising the reaction section, in the knowledge that in the fermentation field the reaction sections (the bioreactor(s)) operate by being closed, sealed, without the ingress of external atmosphere.

The term “microorganisms” is understood to be organisms capable of converting molecules into other molecules of interest by fermentation. In the description that follows, these may also be denoted as “cells” or else as “bacteria”.

The term “living” has the same meaning in the present text as “viable” where the microorganisms are concerned: it relates to microorganisms that are considered to be active with respect to the fermentation.

The term “in suspension” has the same meaning as the term “free”, and denotes cells which are in suspension in the liquid phase as opposed to the cells immobilized on the substrate.

The choice has thus been made according to the invention to control the production of alcohol(s) depending on the living microorganisms on the support, which is the most reliable way to do this, most especially at the start-up of production. Specifically, when starting up production, the first step consists, in the bioreactor, of arranging supports, which are for example porous like polymer foams, and then depositing the microorganisms by injecting a fluid containing microorganisms, and referred to as a preculture, into the bioreactor. These microorganisms come to gradually, in part, colonize the supports by creating biofilms, which are a mixture of microorganisms and extracellular polymers. Another portion of the microorganisms will remain in the liquid phase in the bioreactor. The growth of the biofilms is difficult to control, and the portion of living microorganisms that they contain even more so.

The microorganisms, both in suspension and immobilized, are active with respect to the conversion of the sugars to alcohols; however, the microorganisms in suspension are liable to be washed out (that is to say withdrawn with the alcohols during the extraction thereof from the bioreactor). In addition, it has been found that the immobilized microorganisms that are living themselves also produce butanol, which is a compound that inhibits the growth of the microorganisms.

Specifically, as the biofilm grows, taking into account the increase in the quantity of immobilized living microorganisms, the parameters of the process have to be changed, and in general the incoming flow rate of the reactants into the reactor (that of the fluid containing the sugars to be converted), and the outgoing flow rate of the reaction products from the reactor (that of the fluid containing the alcohols obtained by the fermentation of the sugars), will be increased. The choice is thus made, for controlling the process, and therefore, in particular changing these incoming/outgoing flow rates, to evaluate the quantity of immobilized living microorganisms. But the core of the invention is that this monitoring be done without intervention on the supports, that is to say, specifically, without having to open the bioreactor in order to carry out manipulations or treatments, in particular without sampling portions of support. Now, it is extremely advantageous to perform this evaluation without opening the bioreactor or disrupting its operation. This is because these bioreactors generally operate under sterile and anoxic conditions: any opening of the reactor, any sampling of support in particular, is very complex or even impossible to achieve for succeeding in maintaining these sterile and anoxic conditions, most especially on the industrial scale.

To do this, according to the invention, the quantity of living microorganisms immobilized on the support is monitored on the basis of analyses performed both on the liquid phase of the reaction section and on the fermentation gas(es) produced.

The invention has thus developed indirect monitoring of the quantity of immobilized living microorganisms, by coupling:
measurements on the liquid phase (which may easily be sampled from the bioreactor, or even online), which will make it possible to determine the quantity of living microorganisms in suspension in the reactor,
and measurements on the fermentation gases (which may also be easily sampled from the reactor), which will make it possible to estimate the total quantity of living microorganisms, that is to say the sum of the living (active) microorganisms in suspension and immobilized.

With this coupling of data, it is then possible to deduce therefrom an estimation of the quantity of immobilized living microorganisms, and to do so without touching the support, without opening the bioreactor, and without modifying/disrupting the operation of the bioreactor. With this estimation, the invention makes it possible to modulate (increase) the incoming/outgoing flow rates very precisely in order to take into account and counteract the formation of inhibitory butanol as exactly as possible. This estimation can also make it possible to control the production according to criteria other than the formation of inhibitory compounds: it can make it possible for example to evaluate the saturation of the supports with the microorganisms. This is because, beyond a certain period of time, the support is to a great extent colonized by microorganisms, and clogging phenomena appear: when the support material is in the form of blocks or particles, clogging can be observed between the particles/blocks and/or within the particles/blocks when the material thereof is porous, which then causes production to drop. In addition, it is necessary to take into account the mortality of the cells in the biofilm, the saturation observed therefore being the combination of increasing clogging phenomena and the increasing death of the bacteria over time. The invention thus makes it possible to evaluate the progressive reduction in living microorganisms, and therefore the increase in the quantity of dead microorganisms, and thus to aid in the decision to change some or all of the supports.

Preferably, the analysis on the liquid phase of the reaction section comprises a measurement of the viability of the microorganisms in suspension in the liquid phase of the reaction section.

Advantageously, the viability of the microorganisms in suspension in the liquid phase of the reaction section is measured by flow cytometry on a sample of said liquid phase.

Flow cytometry (FC) is a technology allowing the individual analysis of cells. The cells are aligned according to the principle of hydrodynamic focusing before passing in front of a laser beam. The optical phenomena generated allow an analysis of the physical characteristics (size, structure) of the cells or biological characteristics after incubation with the usual reagents.

This analysis technique is of great interest in the context of the invention insofar as, firstly, it is capable of targeting the measurement of the quantity of the living microorganisms, and secondly because it makes it possible to rapidly obtain the results, in particular in less than 1 hour (15 or 30 minutes, for example).

Preferably, the sample analysed is taken from the reaction section or from the stream of fermentation must leaving the reaction section, the samples preferably being taken according to a fixed or varying frequency. It is thus possible to choose a higher frequency on start-up of production, and a lower frequency thereafter. The frequency of the sampling and of the associated measurements may for example be of the order of around ten minutes, of an hour or of a day, depending on the progress of the production run. The analysis can also be carried out continuously, online, on the stream of fermentation must leaving the reaction section, and may be automated.

The analysis on the fermentation gas(es) produced preferably comprises a measurement of the flow rate thereof at the outlet of the reaction section. This is because, in a bioreactor, there is generally provision in the upper portion, at the level of the gas headspace, of an outlet equipped with a valve that easily makes it possible to recover a sample or measure the outgoing flow rate of gas, knowing that the fermentation produces gases, generally a mixture of hydrogen and carbon dioxide H₂/CO₂. Evaluating the quantity of fermentation gas produced makes it possible to estimate the total quantity of living microorganisms in the bioreactor (in suspension and immobilized).

According to the invention, the analysis on the liquid phase of the reaction section is thus performed to determine the concentration of viable microorganisms of said liquid phase, the analysis (in particular the flow rate analysis) on the fermentation gas(es) produced is performed to evaluate the total concentration of viable microorganisms of the reaction section, and an evaluation of the concentration of the viable microorganisms immobilized on the support is deduced from these analyses. The concentration is understood to be the quantity of cells/microorganisms per ml of reaction volume in the bioreactor.

It will thus be possible, according to the invention, to modulate the incoming flow rate of sugary stream into the reaction section and/or the outgoing flow rate of must from the reaction section according to the monitoring of the quantity of living microorganisms immobilized on the support, which amounts in fact to modulating the rate of dilution of the fermentation in the bioreactor.

As indicated above, the microorganisms form a progressively growing biofilm on the support, and the growth of said biofilm on the support can be evaluated according to the monitoring of the quantity of living microorganisms immobilized on the support, which can yield information of interest at the start of production (for monitoring the initial attachment of the microorganisms on the support), and further on in the production run: it is specifically possible to at least in part evaluate the saturation level of the support according to the monitoring of the growth of said biofilm.

Advantageously, according to the invention, a fermentation must comprising isopropanol, butanol and ethanol is produced, the microorganisms being derived from a strain belonging to the genus Clostridium.

A subject of the invention is also a system for producing alcohol(s) from a fluid containing a sugary carbon-based substrate, in order to produce, by fermentation under the action of microorganisms, a must enriched in alcohol(s) and one or more fermentation gases, implementing the process described above.

A subject of the invention is also a system for producing alcohol(s) from a sugary fluid, in order to produce, by fermentation under the action of microorganisms, a must enriched in alcohol(s) and one or more fermentation gases, such that said system comprises:
a reaction section comprising a support, in particular in the form of polyurethane-type polymer foam, on which microorganisms are immobilized, said reaction section being provided with an inlet for the sugary fluid and an outlet for the must,
means for monitoring the quantity of living microorganisms immobilized on the support without intervention on said supports.

Preferably, the monitoring means comprise:
a flow cytometry device which measures the viability of the microorganisms in suspension in the liquid phase of the reaction section on the basis of samples taken from the reaction section,
a device for measuring the flow rate of the fermentation gas(es) produced at the outlet of the reaction section,
calculation means (any electronic/computer means) which deduce, from the cytometry and gas flow rate measurements, the quantity of living microorganisms immobilized on the support.

The reaction section according to the invention preferably comprises one or more bioreactors operating under sterile and anoxic conditions.

The invention will be described in more detail hereinafter and will be illustrated with the aid of example(s) and figures which do not limit said invention.

### LIST OF FIGURES

FIG. 1 highly schematically represents a bioreactor implementing the invention.

FIG. 2 highly schematically represents the block diagram of the process according to the invention.

FIG. 3 is a graph representing the (fermentation) gas flow rate as a function of the concentration of living microorganisms (in “active” cells).

FIG. 4 is a graph representing the change in the concentration of living microorganisms (“active” cells) as a function of time.

FIG. 5 is a graph representing the estimated concentration of living microorganisms (in active cells) as a function of the same concentration measured experimentally.

FIG. 6 is a graph representing the variation in concentration of living microorganisms immobilized on support as a function of time, as deduced according to the process of the invention.

### DESCRIPTION OF THE EMBODIMENTS

The invention is of interest more particularly for IBE- or ABE-type fermentations with immobilized cells, an example of which is described in the abovementioned patent FR-3 086 670 to which reference can be made.

Continuous IBE or ABE fermentation processes with immobilized cells are employed in order to produce isopropanol and butanol from renewable resources. The bacteria employed in these processes are specifically capable of degrading certain simple sugars in order to form butanol, isopropanol and also gas in the form of an H₂/CO₂ mixture.

The toxicity of butanol and the low growth rate of the bacteria in solventogenic phase limit the performance of the batch and simple continuous process. The use of a continuous process with immobilized cells enables a significant increase in the volume productivity, since the residence time of the bacteria under these conditions is decorrelated from the hydraulic residence time of the bioreactor under study. Moreover, the concentration of bacteria increases in the bioreactor.

The bacteria are immobilized in the form of a biofilm on a solid support. This biofilm is composed of a mixture of cells and extracellular polymers. The process of biofilm formation is difficult to control and depends on numerous factors. Specifically, the hydrodynamic conditions in the bioreactor, the physicochemical properties of the supports used and also the number thereof can influence biofilm development. The proportion of viable, and hence metabolite-producing, cells within this structure can also vary widely depending on the age of the biofilm under study and also on the operating conditions of the process.

It should be noted that the active biomass is located both in suspension in the liquid medium and on the solid supports. Thus, the monitoring of the total quantity of active cells of the biofilm within the bioreactor is a key parameter to know in order to optimally control the process. This is because viable immobilized cells produce butanol, a compound which inhibits bacterial growth. Consequently, the incoming and outgoing volume flow rate of the process needs to be increased as the biofilm grows within the supports, based in the following examples on polyurethane foam.

However, sampling of solid supports cannot be performed in the course of the fermentation processes with immobilized cells. This is because sampling of the support could cause problems as regards the maintenance of the sterility and anoxic conditions of the process. Consequently, a method for indirectly monitoring the development of the biofilm/of the quantity of immobilized active cells in these biofilms is entirely of interest.

The implementation of the invention described below is thus a method for measuring gas flow rate online coupled with measuring the concentration of active cells in suspension by flow cytometry, in order to estimate the quantity of active cells immobilized on the solid support (biofilm).

Specifically, the gas flow rate measured at the reactor outlet is correlated with the total concentration of active cells measured by flow cytometry (see equation below). Thus, when the concentration of active cells in the liquid medium is measured via this same method, the proportion of immobilized active cells can be estimated. The present invention thus makes it possible to estimate the development of the biofilm during start-ups of fermentation without however sampling solid supports, which represents an advantage for the management of the start-up of this process.

$${{\underbrace{rG}}_{gas production rate}}_{( {L}_{gas} \cdot {h}^{- 1} )} = {{\underbrace{{q}_{G}}}_{specific cellular productivity}}_{( {L}_{gas} \cdot {cells}^{- 1} \cdot {h}^{- 1} )} \cdot {{\underbrace{{X}_{v}}}_{active cells}}_{( cells )}$$

The invention may be implemented in the course of a continuous IBE or ABE fermentation with immobilized cells. Over the course of this type of fermentation, the cells are adsorbed onto a porous support, preferentially polyurethane foams. The cells multiply within this porous support as the fermentation continues.

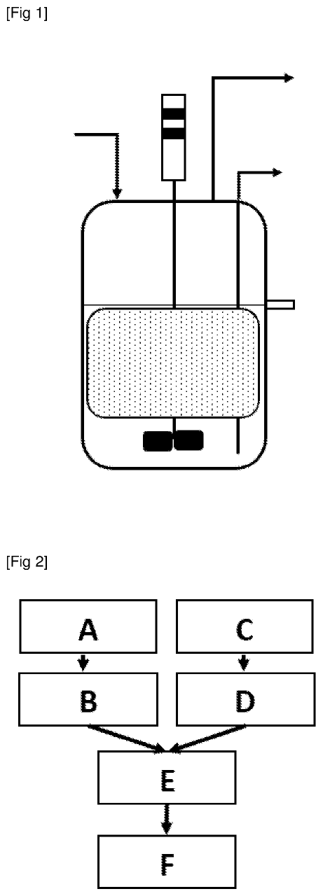

FIG. 1 is a highly simplified representation of a bioreactor 1 capable of implementing the invention. It does not detail all of the conventional equipment of the bioreactors, and is not necessarily to scale. The bioreactor 1 delimits an inner volume substantially in the form of a vertical cylinder, divided into a lower volume V1 filled with a liquid phase and an upper volume V2 which is the gas headspace of the reactor. The bioreactor 1 comprises an inlet fed with a stream of fluid containing the sugary carbon-based substrate_2, and an outlet 3 from which the fermentation must 3 enriched in alcohols is withdrawn. The volume V1 is provided with polyurethane foam support(s) 4. The bioreactor 1 is provided with means 5 for stirring the liquid phase, an outlet 5 allowing samples of liquid phase to be taken with a view to carrying out flow cytometry analyses on them, and an outlet 6 in the volume V2 allowing samples of the gas phase to be taken or the outgoing flow rate of the gas phase to be measured.

The process of the invention can be summarized in the form of a block diagram represented in FIG. 2.
step A: the flow rate of (fermentation) gas leaving the gas headspace V2 (outlet 6, provided with a valve and equipped with conventional flow rate measurement means), in the following examples a gas flowmeter or volumetric meter is used, is measured, giving a gas volume flow rate value of an H₂/CO₂ mixture. The flow rates measured may vary between 0 and 5 L·Lreactor⁻¹·h⁻¹, but are preferentially between 0.5 and 3 L·Lreactor⁻¹·h⁻¹,
step B: an estimation of the total active cells (in suspension in the liquid phase and immobilized on the supports 4 in the volume V1) is deduced therefrom, as detailed hereinafter,
step C: the viability of the cells in suspension in the liquid phase V1 is analysed by taking samples of the liquid phase (outlet 5), the analysis being performed by flow cytometry. This analysis may be automated. The method employed in the examples below is as follows: The viability is measured by diluting a cell suspension sampled from the bioreactor in Mcllvaine buffer until a concentration of between 5.10⁵ and 2.10⁶ cells·mL⁻¹ is obtained. Once this concentration has been reached, propidium iodide and carboxyfluorescein diacetate are added to the cell suspension to a final concentration of 10 μg·mL⁻¹. The cell suspension is then analysed with the aid of a flow cytometer equipped with an argon laser (λ: 488 nm). Light scattered at angles and within the axis of the laser is collected and analysed for each detected particle. The light scattered perpendicularly to the axis of the laser passes through a bandpass filter (530±15 nm) to collect the green fluorescence and also through a high-pass filter (>630 nm) to collect the fluorescence in the red. Flow cytometry, in contrast to the measurement of the dry mass concentration, makes it possible to measure active cells from among the total biomass, which makes it possible to calculate the specific productivities of the process with greater precision,
step D: the number of active cells in suspension in the liquid phase is deduced therefrom,
step E: from the results of steps C and D, an estimation of the concentration of active cells immobilized on the supports 4 is deduced,
step F: the results of step E are utilized for the performance of the fermentation process (choice of the incoming 2 and outgoing 3 flow rates in particular).

This process is iterative, at a given frequency which may change over the course of the fermentation process.

The type of sugary carbon-based stream, the type of microorganism and the type of support that may be used are described hereinafter.

The fluid containing the sugary carbon-based substrate 2.

According to one or more embodiments, the fluid containing the sugary carbon-based substrate comprises an aqueous solution of C5 and/or C6 sugars obtained from lignocellulose, and/or of sugars obtained from sugar-producing plants (for example, glucose, fructose and sucrose), and/or of sugars obtained from starchy plants (for example, dextrins, maltose and other oligomers, or even starch). According to one or more embodiments, the aqueous solution of C5 and/or C6 sugars originates from the treatment of a renewable source. According to one or more embodiments, the renewable source is of the lignocellulosic biomass type which may notably comprise ligneous substrates (for example, deciduous plants and coniferous plants), agricultural byproducts (for example, straw) or byproducts from industries generating lignocellulosic waste (originating from agrifood or paper industries). The renewable source may also originate from sugar-producing plants, for instance sugar beet and sugarcane, or from starchy plants such as corn and wheat. The aqueous solution of C5 and/or C6 sugars may also originate from a mixture of various renewable sources. According to one mode of execution, this solution is sterilized for 20 minutes at 120° C.

The biomass produced by the strain belonging to the genus Clostridium.

The bacterial biomass is mainly adsorbed in the form of a biofilm onto a solid support. Preferably, the bacteria are strains belonging to the species Clostridium beijerinckii and/or Clostridium acetobutylicum. The bacteria used in the process may be strains which may or may not be genetically modified and which naturally produce isopropanol and/or Clostridium strains which naturally produce acetone and which are genetically modified to make them produce isopropanol. In the following examples, it is Clostridium beijerinckii DSM 6423.

### The Solid Support

The solid support comprises a polyurethane foam. Polyurethane foam is particularly advantageous since it allows access not only to the production of mixtures of IBEA type, but also allows access to production of continuous type by immobilization of the bacterial biomass. Specifically, the polyurethane foam is capable of fixing bacteria of the genus Clostridium in a sufficiently substantial manner (i.e. beyond the dilution rate causing cell wash-out) making it possible to continuously produce mixtures of IBEA type. Furthermore, polyurethane foam is suitable for immobilization by immersion in a reactor. Alternatively, a foam based on ceramic material(s) can be used.

According to one or more embodiments, the polyurethane foam has:
volume cavities (i.e., pores or cells) the equivalent sphere diameter of which is between 0.1 and 5 mm, preferably between 0.25 mm and 1.1 mm, preferably between 0.55 and 0.99 mm, and/or
an apparent density (i.e. apparent mass per unit volume) measured in air of between 10 and 90 g/L, preferably between 10 and 80 g/L, preferably between 15 and 45 g/L, such as between 20 and 45 g/L or between 25 and 45 g/L.

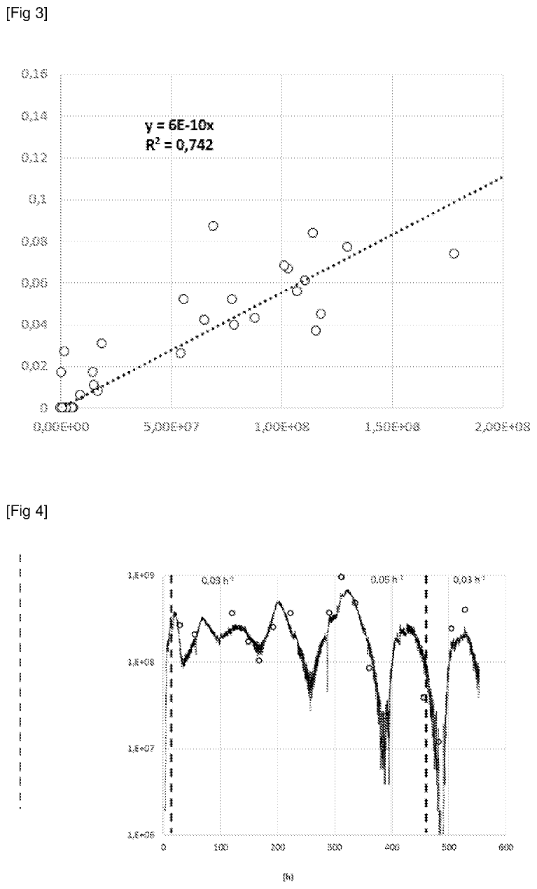

In the case of a batch fermentation with Clostridium beijerinckii DSM6423, the correlation between active cells in suspension and the fermentation gas flow rate was first established and is presented in FIG. 3, which is a graph representing the concentration of active cells in cells·mL⁻¹ on the x axis, and the flow rate of gas leaving the bioreactor in L·h⁻¹ on the y axis.

These tests were carried out on GAPES medium, at 34° C. and without pH regulation.

The operating conditions for these tests are as follows:

After reduction of the CALAM medium, the composition of which is given in table 1 below, the spores are activated by heat shock at 100° C. for one minute, and the medium is inoculated to 2%. The preculture is then incubated at 34° C., under anaerobic conditions and with stirring at 100 rpm for 24 h.

TABLE 1

| Component | Concentration (g.L-1) |
|---|---|
| Potato | 250 |
| (NH₄)₂SO₄ | 2.0 |
| CaCO₃ | 2.0 |
| Glucose | 10.0 |

A second preculture in GAPES medium, the composition of which is given in table 2 below, is then carried out prior to the final seeding of the fermenter. This time, the GAPES medium is inoculated to 10% and then the preculture is incubated at 34° C., under anaerobic conditions and with stirring at 100 rpm for 24 h.

TABLE 2

| Component | Concentration (g.L-1) |
|---|---|
| FeSO₄, 7H₂O | 0.0066 |
| MgSO₄, 7H₂O | 1.0 |
| KH₂PO₄ | 1.0 |
| K₂HPO₄ | 0.6 |
| CH₃COONH₄ | 2.9 |
| p-Aminobenzoic acid | 0.1 |
| Granulated yeast extract | 2.5 |
| Glucose | 45 or 60 |

Lastly, fermentation in the reactor is carried out with GAPES medium, which has been reduced beforehand, with an inoculation to 10%. The culture conditions employed are: temperature at 34° C., stirring of 200 rpm, without nitrogen bubbling and without pH regulation.

In the case of continuous fermentations, a first batch growth phase takes place for 8 h and then the reactor is connected to a feed bottle (GAPES medium) and a withdrawal bottle. The feed is started and gradually increased.

The gas flow rate was measured with the aid of a Ritter volumetric meter and the quantity of active cells was measured by labelling with cFDA and analysis by flow cytometry.

The measurement protocol is as follows:

Mcllvaine buffer pH 4
1. Prepare a 0.1 M (i.e. 19.21 g/L) citric acid solution and a 0.2 M (i.e. 28.38 g/L) Na₂HPO₄ solution.
2. To obtain a pH equal to 4, mix 307.25 mL of the 0.1 M citric acid solution with 192.75 mL of the 0.2 M Na₂HPO₄ solution. Verify the pH of the buffer solution obtained.
3. Filter the solution and store it at 4° C.

### Viability Label

Propidium iodide: 1.4986 mM in milliQ water—store at 4° C.
Chemchrome V8 sold by Biomérieux (cFDA: carboxyfluorescein diacetate): 0.217 μM in acetone-store at −20° C.

### Cell Labelling

1. Dilute the cell suspension in Mcllvaine buffer to obtain a concentration of around 1.106 cell/mL.
2. Add 10 μL of Chemchrome V8 and 10 μL of propidium iodide to 1 mL of the diluted cell suspension.
3. Incubate for 10 min at 40° C.
4. Run the sample through the flow cytometer.

FIG. 3 is a graph representing the (fermentation) gas flow rate as measured, expressed in L·h⁻¹, as a function of the concentration of living microorganisms (in “active” cells), expressed as number of viable cells per ml of volume V1 (working volume) of bioreactor. This figure demonstrates that a linear relationship between gas flow rate and concentration of active cells measured by flow cytometry is obtained. The latter thus makes it possible to estimate the specific cellular productivity of fermentation gas according to the following relationship:

$${{\underbrace{rG}}_{gas production rate}}_{( {L}_{gas} \cdot {h}^{- 1} )} = {{\underbrace{{q}_{G}}}_{specific cellular productivity}}_{( {L}_{gas} \cdot {cells}^{- 1} \cdot {h}^{- 1} )} \cdot {{\underbrace{{X}_{v}}}_{active cells}}_{( cells )}$$

The value of the parameter qc corresponds in fact to the slope of the line of regression: 5.54·10⁻¹⁰±3.5·10⁻¹¹ L·cells⁻¹·h⁻¹. Assuming that the specific cellular productivity q_{G} is constant over time, this same relationship can then be used to calculate the total quantity of active cells from the gas flow rates measured during continuous fermentation. This method was thus used in a second phase to estimate the concentration of active cells over the course of a continuous fermentation with free cells.

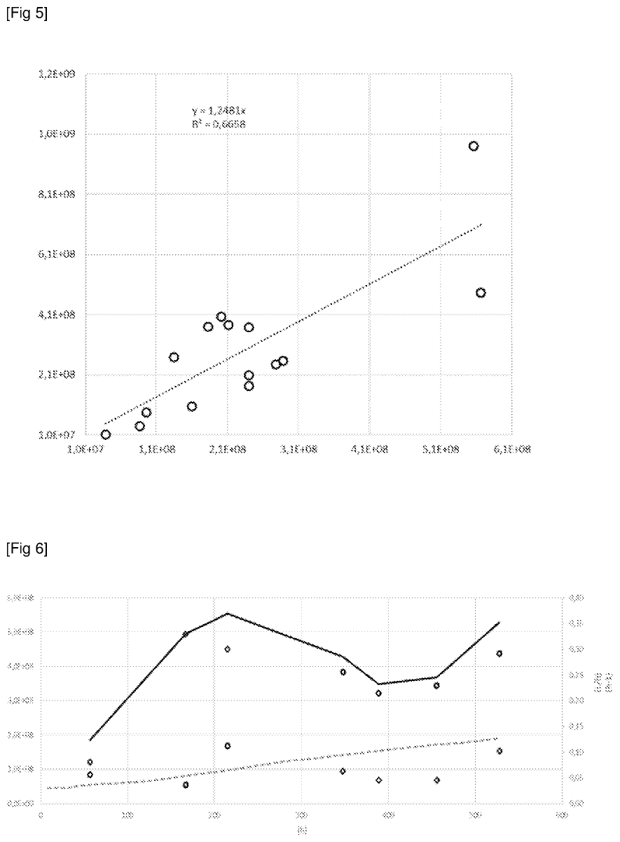

The results are illustrated in FIGS. 4 and 5:
FIG. 4 is a graph representing time expressed in hours on the x axis, and the concentration of active cells over the course of an IBE fermentation at 34° C. with free cells, expressed as active cells·mL⁻¹, on the y axis.
FIG. 5 is a graph representing the concentration of active cells estimated over the course of an IBE fermentation at 34° C. with free cells, expressed as active cells·mL⁻¹, on the x axis, and the active cells measured experimentally, in the same units, on the y axis.

The results of the estimation of the concentration of free cells are presented in FIG. 4. The continuous plots represent the estimation of the concentration of active cells over time on the basis of the measured gas flow rate.

The white points in FIG. 5 represent the concentration of active cells measured by flow cytometry: FIG. 5 shows that the data estimated on the basis of the specific cellular productivity q_{G} calculated experimentally over the course of the batch fermentations are close to the experimental data (R²=0.87). This test makes it possible to validate the assumption of constancy of the specific cellular productivity q_{G} (5.54·10⁻¹⁰ L·cells⁻¹·h⁻¹).

It is therefore quite possible to estimate the concentration of total active cells over the course of the fermentation. This relationship can then be used inventively in order to estimate the concentration of active cells in the biofilm of a continuous process with immobilized cells, provided that the specific cellular productivity of gas of the immobilized cells is equal to that measured in suspension.

$${{\underbrace{rG}}_{gas production rate}}_{( {L}_{gas} \cdot {h}^{- 1} )} = {{\underbrace{{q}_{G}}}_{specific cellular productivity}}_{( {L}_{gas} \cdot {cells}^{- 1} \cdot {h}^{- 1} )} \cdot {{\underbrace{( {X}_{v}} suspension + Xv biofilm )}_{total active cells}}_{( cells )}$$

Thus:

$${X}_{v} biofilm = \frac{rG}{{q}_{G}} - {X}_{v} suspension$$

In this case, a measurement of the quantity of active cells in suspension by flow cytometry is carried out. It is thus possible to monitor the colonization of the biofilm without sampling and analyzing solid supports.

The advantage of this invention is that it makes it possible to implement the dilution rate of the fermentation in order to maximize the concentration of immobilized active cells, without invasive sampling in the bioreactor during production. The process developed within the context of the present invention thus makes it possible to limit the bias introduced by the sampling of the solid supports during the studying of the kinetics of Clostridium (C. beijerinckii or acetobutylicum) growth in a biofilm: loss of sterility (that is to say risks of contamination) and of anoxic conditions (ingress of oxygen) affecting the growth of the bacteria under study.

This process can be extrapolated/applied analogously to other types of fermentations with immobilized cells, as long as a single type of bacterial strain is used at the least, and as long as the bacterial strains employed possess a specific productivity of gas that is fixed or changes little throughout the fermentation under study.

### Example 1

The system described is composed of the bioreactor 1 with a working volume of 0.35 L. The plate of this bioreactor is equipped with an inlet for the feeding of substrate 2, a dip tube for taking samples, the outlet of liquid is provided by an overflow system. The polyurethane foams 4 are placed in the bioreactor 1 so as to have 0.24 L of foam per liter of bioreactor. The system is sterilized under vacuum at 120° C. for 20 min. A volume of 0.315 L of sterile and anoxic carbon-based substrate (GAPES medium) is introduced into the bioreactor. The feed vessel (not shown in FIG. 1) containing sterile carbon-based substrate (GAPES medium) is connected to the bioreactor 1. The fermentation is performed by the strain Clostridium beijerinckii DSM6423. The inoculation is performed from 0.035 L of preculture of this strain that has been incubated beforehand for 24 h at 34° C. and under anoxic conditions.

Once the inoculation has been performed, a batchwise incubation period of 8 h is observed. The test was carried out at 34° C., with stirring of 200 rpm, and the pH of the fermentation is not controlled. The carbon-based substrate 2 is then fed continuously to the reaction portion V1 of the bioreactor 1 with the aid of a peristaltic pump. The incoming volume flow rate of the carbon-based substrate 2 and the outgoing volume flow rate of the fermented liquor, also called fermentation must 3, are equal throughout the test. The flow rate normalized by the working volume of the bioreactor 1 is called the dilution rate. As indicated in FIG. 1, the latter is increased as the test progresses in order not to accumulate inhibitory concentrations of butanol as the biofilm grows. The flow rate of gas produced by the strain used is measured with the aid of a volumetric meter.

FIG. 6 represents, on the x axis, the time expressed in hours and, on the y axis, along the left-hand vertical axis, the concentration of living microorganisms per ml of reaction volume of the bioreactor, and, along the right-hand vertical axis, the flow rate in liters/hour of the incoming 2 and outgoing 3 streams (which are identical) of the bioreactor.
The diamonds represent the living microorganisms in suspension/ml of volume V1 of the bioreactor,
The circles represent the concentration of living microorganisms on the biofilm/ml of volume V1 of the bioreactor as estimated according to the invention,
The solid-line curve represents the measurement of the fermentation gas flow rate,
The dotted-line curve represents the change in the dilution rate in h⁻¹ as defined above.

As indicated in FIG. 6, the gas volume flow rate measured can serve to estimate the total concentration of active cells in the fermentation medium. It is thus possible, on the basis of the gas measurement, and of the measurement of the viability of the cells in suspension by flow cytometry, to estimate the concentration of viable cells that are immobilized in the bioreactor.

These data show that when the dilution rate applied to the system is low at the start of the test, the majority of the viable cells (that is to say the living microorganisms) are in suspension. When the dilution rate is increased, the viable cells in suspension are washed out of the bioreactor, although the gas volume flow rate remains constant. Counting the number of viable cells in suspension and analysing the fermentation gas volume flow rate then make it possible to verify that the majority of the viable cells are then immobilized on the polyurethane foams. These analyses can then serve to better understand where the viable cells are located in the bioreactor under consideration, and thus to optimize the fermentation process.

It is thus possible to optimally modulate the dilution rate throughout the fermentation process, which is of most particular interest at the start-up of the process.

1. A process for producing alcohol(s), according to which a culture medium containing a sugary carbon-based substrate (2) is introduced into a reaction section (1) comprising a support (4) on which microorganisms are immobilized, in order to produce, by fermentation under the action of said microorganisms, a must (3) enriched in alcohol(s) and one or more fermentation gases, characterized in that the process is operated continuously in liquid phase, and in that, in order to control the production, the quantity of living microorganisms immobilized on the support is monitored without intervention on said supports, on the basis of analyses performed both on the liquid phase (V1) of the reaction section (1) and on the fermentation gas(es) produced.

2. The process as claimed in claim 1, characterized in that the analysis on the liquid phase (V1) of the reaction section (1) comprises a measurement of the viability of the microorganisms in suspension in the liquid phase of the reaction section.

3. The process as claimed in claim 2, characterized in that the viability of the microorganisms in suspension in the liquid phase (V1) of the reaction section (1) is measured by flow cytometry on a sample of said liquid phase.

4. The process as claimed in claim 3, characterized in that the sample analysed is taken from the reaction section (1) or from the stream of fermentation must (3) leaving the reaction section, the samples preferably being taken according to a fixed or varying frequency.

5. The process as claimed in claim 1, characterized in that the analysis on the fermentation gas(es) produced comprises a measurement of the flow rate thereof at the outlet of the reaction section (1).

6. The process as claimed in claim 1, characterized in that the analysis on the liquid phase (V1) of the reaction section (1) determines the concentration of viable microorganisms of said liquid phase, in that the analysis on the fermentation gas(es) produced evaluates the total concentration of viable microorganisms of the reaction section, and in that an evaluation of the concentration of the viable microorganisms immobilized on the support is deduced from these analyses.

7. The process as claimed in claim 1, characterized in that the flow rate of sugary stream (2) entering the reaction section and/or the flow rate of must (3) leaving the reaction section (1) is/are modulated according to the monitoring of the quantity of living microorganisms immobilized on the support.

8. The process as claimed in claim 1, characterized in that the microorganisms form a progressively growing biofilm on the support (4), and in that the growth of said biofilm on the support is evaluated according to the monitoring of the quantity of living microorganisms immobilized on the support.

9. The process as claimed in claim 1, characterized in that the degree of saturation of the support (4) is evaluated according to the monitoring of the growth of said biofilm.

10. The process as claimed in claim 1, characterized in that a fermentation must (3) comprising isopropanol, butanol and ethanol is produced, the microorganisms being derived from a strain belonging to the genus Clostridium.

11. A system for producing alcohol(s) from a fluid containing a sugary carbon-based substrate, in order to produce, by fermentation under the action of microorganisms, a must enriched in alcohol(s) and one or more fermentation gases, characterized in that said system comprises:
a reaction section (1) comprising a support (4), in particular in the form of polyurethane-type polymer foam, on which microorganisms are immobilized, said reaction section being provided with an inlet for the sugary fluid and an outlet for the must,
means for monitoring the quantity of living microorganisms immobilized on the support without intervention on said supports, by analyses performed both on the liquid phase (V1) of the reaction section (1) and on the fermentation gas(es) produced.

12. The system as claimed in claim 11, characterized in that the monitoring means comprise:
a flow cytometry device which measures the viability of the microorganisms in suspension in the liquid phase of the reaction section on the basis of samples taken from the reaction section,
a device for measuring the flow rate of the fermentation gas(es) produced at the outlet of the reaction section,
calculation means which deduce, from the cytometry and gas flow rate measurements, the quantity of living microorganisms immobilized on the support.

13. The system as claimed in claim 11, characterized in that the reaction section (1) comprises one or more bioreactors operating under sterile and anoxic conditions.
